(19) United States
(12) Patent Application Publication
(10) Pub. No.: US 2026/0347741 A1
(43) Pub. Date: Nov. 5, 2026

(54) Single Cell Handling Device

(71) Applicant: Cell Tech Holding Pty Ltd, Victoria (AU)

(72) Inventor: Jeremy Thompson, Victoria (AU)

(21) Appl. No.: 19/123,541

(22) PCT Filed: Oct. 24, 2023
(86) PCT No.: PCT/AU2023/051062

§ 371 Date: Apr. 23, 2025

(30) Foreign Application Priority Data
Oct. 24, 2022 (AU) 2022903148

(51) Int. Cl. C12M 1/26; C12M 1/00; C12M 1/12; C12M 1/32

(52) U.S. Cl. CPC C12M 33/04; C12M 23/06; C12M 23/12; C12M 23/34; C12M 23/38; C12M 23/40; C12M 29/20

(57) ABSTRACT

The field generally relates to devices for handling single cells or small cell masses, in particular, for handling single mammalian cells or mammalian cell masses, methods for their manufacture and methods for their use. Devices are generally single cell handling devices for handling a single cell during a microinjection procedure comprising; a base dish having an upper surface and a lower surface, a microwell array comprising one or more microwells, the one or more microwells comprising; at least one microwell wall defining an upper microwell opening and a central well, the at least one microwell wall having formed therein two convex cell stabilizing protrusions each defining a cavity positioned opposite to the other on a common horizontal plane, and a moat for maintaining fluid therein located at the base dish upper surface and extending around the perimeter of the microwell array.

### TECHNICAL FIELD

The field generally relates to devices for handling single cells or small cell masses, in particular, for handling single mammalian cells or mammalian cell masses, methods for their manufacture and methods for their use. Devices are generally applicable to the field of infertility treatment and assisted reproduction; their uses may include In Vitro Fertilisation (or “IVF”) techniques or more particularly IntraCytoplasmic Sperm Injection (or “ICSI”). However, the single cell handling devices described herein are equally applicable to mammalian cell culture, stem cell differentiation, cell array assays and other infertility treatments.

### BACKGROUND

Advances in microscale tissue culture techniques have given rise to new therapies, products, and processes, many of which are still in their infancy. The potential benefits to the improvements in cellular cultivation and proliferation, at the microscale, are especially significant within the field of reproductive medicine. The nature of the field offers little alternative to in vitro cultivation and manipulation of individual autologous cells which must be harvested, manipulated and reintroduced to the patient.

Reproductive assistance by way of In Vitro Fertilisation (“IVF”) has become more accessible and has seen recent improvement. Consequently, it is being accessed by an increasing number of patients. Data released by the Human Fertilisation and Embryology Authority show that, overall, women starting IVF treatment are more likely to have a child than previously. However, a wide range of variations in the success rates of IVF clinics are observed between individual clinics, with some clinics achieving a success rate as high as 46%, while the rate at others is as low as 10%.

The likelihood of success can vary at several stages in the IVF process; during harvest of the oocyte, fertilisation of the oocyte, development of the early embryo, vitrification of the embryo and transfer of the embryo. The success of processes requiring oocyte manipulation are, currently, largely dependent on the skill of the embryologist.

IntraCytoplasmic Sperm Injection (or “ICSI”) is an assisted reproductive technology procedure conducted within infertility clinics, primarily for the treatment for male factor infertility (Boulet et al. 2015). It is an alternative procedure for fertilisation of an oocyte by a spermatozoon by replacing IVF procedures.

In IVF, a solution of spermatozoa is co-incubated with an oocyte and relies on one of many thousands of spermatozoa to bind to, then penetrate the oocyte, thereby fertilising the oocyte. The ICSI procedure differs by directly injecting a single spermatozoon into the cytoplasm of the oocyte.

The procedure was first described by Palermo et al. (1992) and provides a therapeutic solution for male factor infertility, especially men with oligospermia and azoospermia; where the sperm count within an ejaculated semen sample is too low to provide an optimal concentration of co-incubated motile spermatozoa to maximise the opportunity of fertilisation during IVF.

The ICSI procedure is reliant on tools to manipulate the positioning of both the spermatozoon and oocyte and then injecting the spermatozoon into the oocyte. These tools include two micromanipulators, one used for the positioning of the oocyte, held in place by a finely drawn and heat polished pipette, commonly termed the ‘holding pipette’ and a second for positioning of a finely drawn ‘injection’ pipette that contains the sperm. Both pipettes are attached to a fine-controlled syringe-like ‘injector’. The first controls the degree of negative and positive pressure to hold, position and release the oocyte with the holding pipette. The second is used to control the capture and injection of the spermatozoon within the injection pipette. The process and equipment are well known in the art and are described in detail in Joris et al. (1998).

ICSI and other IVF processes present a risk of injury from the physical manipulation required for introduction of the sperm (whether this is intra-cytoplasmic or not). The risk of injury or shock from manipulation and handling, and therefore the success of ICSI and other IVF procedures, is largely influenced by:
a. the skill and care of the embryologist,
b. the precision of the tools and apparatus that the embryologist has available to them, and
c. the environmental conditions created in the laboratory that are influenced by the embryologist (e.g. sterility, temperature, prevention of any form of contamination, including volatile organic compounds).

The present disclosure addresses the second problem described above; that is, the deficiencies in the precision of the tools and apparatus that the embryologist has available to them.

To compensate for the lack of precision single cell handling devices, positive and negative pressure holding pipettes and injection pipettes are required to pick up, manipulate and place the oocyte.

In use, control of the amount of pressure used for creating a vacuum seal between oocyte and the holding pipette is not quantified by the embryologist. This leads to variations between procedures and between embryologists in the amount of negative pressure applied to the oocyte during an ICSI procedure. Typically, deformation of the oocyte is caused by the suction applied by the holding pipette, causing movement of ooplasm within the pipette.

Negative pressure handling of the oocyte frequently causes invagination of the Zona Pellucida within the suction micropipette, thereby causing stress or damage to the cell.

Invagination of the oolemma (the plasma membrane of the oocyte) refers to the degree of cytoplasmic misshaping caused by the injection pipette being pushed initially through the zona pellucida, then the oolemma. Shear-stress is caused by literally squeezing the oocyte and is a major cause of cellular stress during ICSI procedures.

Oocytes are normally “protected” by a layer of “nurse-cells”, the cumulus oophorus. It is well known that a bi-directional communication system applies to the cumulus and oocyte that determines oocyte quality (Gilchrist et al. 2008). Removal of cumulus before fertilisation is widely known to negatively impact oocyte health (Gilchrist et al. 2008). The ICSI procedure requires the cumulus layer to be removed, for unencumbered microscopic visualisation of the oocyte. This removal of cumulus cells normally occurs following the fertilisation event in a standard IVF treatment.

During ICSI, the oocyte is placed without its protective cell layer prior to fertilisation. The oocyte is then exposed to a number of manipulations that it would not be subject to in IVF, whether it be exposure to microscopic light, the negative pressure applied by a holding pipette to maintain its position, the invagination of the oocyte caused by the point-pressure of the injection pipette against the holding pipette, the withdrawal of cytoplasm into the injection pipette to assess complete insertion and the injection of a synthetic polymer (PVP) used to ‘slow’ the sperm for capture into the injection pipette.

According to Latham (2016), cellular stress often mediates a cell response, characterised by activation of the endoplasmic reticulum stress response, which causes the unfolding of proteins, which in turn leads to cell apoptosis and necrosis.

Although there have been improvements in the functional control of the micromanipulators and injectors, the actual work-flow procedure is the same as first developed in 1992. Furthermore, the procedure requires extensive training to master (Tiegs and Scott, 2020).

Improvements to the ICSI procedure to reduce the technical skill and the time taken for the procedure to be conducted have not emerged. Attempts to improve the results of the ICSI procedure has focussed on either modifying the holding pipette design (Fernandez et al. 2020; Ma et al. 2020) or by introducing an injection system that is reliant on a piezo-motor function (Zander-Fox et al. 2021).

Zander-Fox et al. (2021) have developed a PIEZO-ICSI technique which utilises a piezoelectric actuator to generate high-speed movement of the injection pipette; effectively causing the precise microdrilling of the zona. This technique reduces the need for physical pressure on the oocyte membrane and obviates the needs for cytoplasmic agitation. It is therefore potentially a gentler form of microinjection but does not offer improvement in the handling of the oocyte.

While advances have been made to microinjection devices used in IVF therapies as well as visualisation devices, very little progress has been made for the development of devices for handling the cells involved when they must be manipulated, or when containing them in a fashion that reduces excessive handling or manipulation.

US 2021/0230525 (Alcaide, F. V. 2021) describes a vacuum pipette for retaining oocytes and intracytoplasmic sperm injection methods based on an elongated and narrow cylindrical body and frustoconical reduction portion for retaining the oocyte under vacuum pressure. This vacuum pipette obviates the need for suctioning of the cytoplasm prior to microinjection, however, the vacuum pressure causes deformation of the cell and does not prevent invagination of the oolemma at the point of microinjection.

Similarly, Ma et al. (2020) propose a trumpet shaped variation to the traditional holding pipette utilised in human reproductive technology. This modification also does not resolve the problems of deformation of the cell during microinjection nor invagination of the oolemma at the point of microinjection.

A simpler and more precise oocyte and/or sperm cell handling device is needed that removes the need for pressurised handling devices and ameliorates the problems with pressurised handling devices. Such simple, precision oocyte and/or sperm cell handling device, preferably, should be compatible with precision microinjection techniques, such as PIEZO-ICSI techniques.

Precision tools and equipment reduce the likelihood that an embryologist may make errors resulting in injury or shock to the sperm, oocyte, or embryo. Further, tools or equipment that reduce or eliminate physical interventions or manipulations by the embryologist reduce the likelihood that an event causing physical shock to the sperm, oocyte, or embryo may occur.

A cell handling device is described herein that maintains the physical stability of the sperm, oocyte and/or embryo, optionally, within a dedicated culture environment. The embryologist may more easily and/or accurately handle or manipulate the sperm, oocyte or embryo, without the need for pressurized pipetting devices; and thereby minimise the risk of physical shock and/or biochemical stress to the cell/s. The stabilisation of the cells during culture may improve the success of ICSI procedures.

Additionally, oocytes are sensitive to any variation of environmental factors such as temperature, osmolality, oxygen concentration, nutrient restriction, hyperglycemic conditions, and pH (Latham, 2016). The cell handling device described herein may optionally reduce variation in the optimised ex-vivo environment thereby further reducing biochemical stress to the sperm, oocyte or embryo.

The cost benefits of eliminating the need for vacuum apparatus and simplifying the cell handling processes arise from reduced labour costs, reduced capital expenditure and reduced training time (Yagoub et al. 2022). This not only improves the profitability of clinics but improves the accessibility of ICSI technology to less affluent communities. For new clinics, improved cells handling processes reduce the cost burden of traditional equipment required for performing ICSI potentially benefitting both the clinic and patient.

The cell handling devices described herein may not only resolve difficulties in the successful fertilisation and culture of developing embryos but may also be applicable to the culture of other cell lines sensitive to changing the culture environment in a gradient process.

### SUMMARY

In a broad aspect, embodiment described herein relate to a single cell handling device for handling a single cell during a microinjection procedure comprising; a base dish having an upper surface and a lower surface, a microwell array comprising one or more microwells, the one or more microwells comprising; at least one microwell wall defining an upper microwell opening and a central well, the at least one microwell wall having formed therein two convex cell stabilizing protrusions each defining a cavity positioned opposite to the other on a common horizontal plane, the cell stabilizing protrusions comprising a first cell stabilizing protrusion defining a channel opening at a perimeter of an access channel extending outwardly through the microwell wall, and the second cell stabilizing protrusion defining a vent opening, the access channel having a channel base aligning with a centre point of the second cell stabilizing protrusion at a horizontal plane, and a moat for maintaining fluid therein located at the base dish upper surface and extending around the perimeter of the microwell array.

The moat of the single cell handling device is preferably in fluid exchange with the one or more microwells via the access channel, the moat having an inner wall proximal to the microarray and an outer wall distal to the microarray, the outer wall extending vertically to a height taller than the channel base of the access channel and the vent opening is entirely located higher than the channel base of the access channel.

The first cell stabilizing protrusion and the second cell stabilizing protrusion are preferably elliptically shaped in a horizontal plane. Preferably, a diameter of the first cell stabilizing protrusion and the second cell stabilizing protrusion is in the range of 10 microns to 500 microns. Alternatively, a diameter of the first cell stabilizing protrusion and the second cell stabilizing protrusion is in the range of 100 microns to 200 microns. Protrusion sizes may be adapted to accommodate the range of sizes of various cell or cell masses.

Preferably, the at least one microwell wall is cylindrical in shape and the one or more microwells comprise a microwell base, wherein the at least one cylindrical microwell wall and the microwell base define the central well.

In further aspects, the at least one cylindrical microwell wall has a diameter greater than the diameter of the cell stabilizing protrusions. In particular, the at least one cylindrical microwell wall has a diameter in the range of 0.1 microns to 1000 microns; alternatively, the at least one cylindrical microwell wall has a diameter in the range of 200 microns to 300 microns.

Preferably, the access channel comprises two parallel channel walls, a channel base and an upper opening.

Preferred single cell handling devices comprise a cover and one or more stacking connectors, located at an upper surface and at a lower surface so as to connect multiple base dishes when stacked.

In various embodiments, the sample support platform at the upper surface of the base plate has a sampling edge proximal to the access channel wherein the sampling edge is stepped or sloped at a gradient to align with the horizontal plane of the channel base.

Method of use of the single cell handling device are preferably for performing an intracytoplasmic sperm injection procedure comprising the steps of;
obtaining a single cell handling device according to claim 1,
adding culture media to the moat of the single cell handling device,
obtaining an oocyte and a sperm sample,
adding the oocyte to the microwell central well,
placing the sperm sample on the sample support platform,
obtaining a microinjection apparatus,
aspirating a sperm from the sperm sample through a tip of the microinjection apparatus,
placing the tip through the access channel of the microwell,
shifting the oocyte to the second cell stabilizing protrusion with the tip,
microinjecting the oocyte with the sperm,
withdrawing the microinjection tip by cradling the oocyte in the first cell stabilizing
protrusion while solely removing the microinjection tip.

Preferred methods of manufacture of single cell handling devices may comprise the fabrication of the base dish and fabricating the microarray and the moat thereon. Alternative methods of manufacture may comprise the fabrication of the microarray and the moat within the base dish.

Preferred methods of manufacture comprise a two part fabrication comprising a two photon polymerised printed part inserted into a polystyrene injection moulding.

In a broad aspect, various embodiments relate to a single cell handling device comprising; a microwell being formed from at least one microwell wall, having two opposing access apertures located therethrough.

The single cell handling device is preferably configured to be formed at a microscale. It is also therefore, preferably formed from materials suitable for microscale production which are non-toxic to cells.

Preferably, the microwell wall comprises an exterior wall surface having formed therein two or more microwell hemispheres. In an alternate form, the exterior wall surface may have formed therein a single microwell hemisphere spanning the circumference of the microwell wall. Preferred microwell hemispheres are shaped to provide optimal mechanical support for the cell maintained therein. Accordingly, the microwell hemispheres may be formed in various configurations and sizes. For example, a hemisphere may have a circular hemispherical shape (e.g. a hemispherical cylinder), an oval hemispherical shape (e.g. a spheroid), a profile with a conic section, or a parabolic profile (e.g. an elliptical paraboloid). The profile of the hemisphere may be graded to increase along a hemispherical groove formed within the exterior wall surface of the microwell.

Preferably, the diameter of a microwell hemisphere is sized to suit the size of a cell maintained therein. For example, an oocyte including the zona pellucida is approximately between 165 microns to 150 microns; therefore preferably, a micro hemisphere is sized approximately between 50 microns to 300 microns for all mammalian oocytes (including those of non-human origin), approximately between 100 microns to 200 microns specifically for human oocytes, or approximately between 10 microns to 500 microns for embryos and other cells; all being of a configuration able to accommodate oocyte diameter ranges from 165 microns to 150 microns.

Preferably, the two opposing access apertures are formed through the microwell wall within a microwell hemisphere. Accordingly, in certain embodiments two microwell hemispheres may be located so as to be opposing one another. Alternatively, at least one microwell hemisphere may span a sufficient length around the microwell wall circumference so as to enable two access apertures to be formed through the microwell hemisphere and also be located opposite one another.

Design choices for forming microwell hemispheres will be directed to those providing greatest support for the oocyte during injection and retrieval of the injection pipette during an ISCI procedure.

Access apertures may form a channel within the microwell wall between the internal surface of the microwell wall and the external surface of the microwell wall. Preferred channel configurations and sizes may be designed and selected on the basis of benefits provided to the embryologist in handling and manoeuvring the injection pipette. Preferred channels may have a closed profile or an open profile. They may have a simple profile, for example a circular hole or an oval slot. Or they may have a complex profile, for example a T-shape or crosshair shaped profile.

The channel may further be shaped so is to assist with clearing air pockets or bubbles when the embryologist is loading the microwell with media.

Alternatively, the channel may be shaped to assist with the movement of media for balancing fluidic pressure, for example to avoid back pressure behind the cell when it is being manipulated. This is particularly important to avoid back pressure behind an oocyte when an embryologist is performing an ICSI procedure.

Preferably, the microwell is open from above to enable access to the microwell for addition of media, or to access and retrieve oocytes. Further, one or more internal surfaces of the microwell are preferably curved.

In further broad aspects, embodiments described herein relate to cell handling arrays comprising one or more microwells.

Preferred cell handling arrays comprise two or more microwells as described herein. Preferably, the two or more microwells are located at a fixed position relative to one another for easy operator navigation of the array. Microwells may be positioned within an array to form various configurations, or to form different pitches of the fixed relative positions. For example, microwells may form a linear array, a circular array, a grid array, other shapes or a combination of any or other shapes and configurations.

In a further preferred form, the single cell handling device or cell handling microarray is located within a base well. The base well is preferably formed of at least one side wall and at least one base wall. The at least one side wall and at least one base wall form a container to contain the single cell handling device or cell handling microarray and fluid media therein. The single cell handling device or cell handling microarray may further, preferably, be located within a larger well for the additional containment of media and or oil used in the performance of ICSI procedures.

Preferred larger wells may be specifically adapted for use with the single cell handling device or cell handling microarrays described herein. Alternatively, they may be “off the shelf” cell culture products, which may or may not be specifically designed for the performance of ICSI procedures.

Preferred base wells comprise one or more sperm platforms, more preferably, two sperm platforms. Preferably, single cell handling devices are arranged in a cell handling microarray located on a base wall of a base well, and two linear sperm platforms are also located on a base wall of a base well, substantially parallel with and adjacent to the cell handling microarray.

Preferred sperm platforms comprise an uppermost surface configured in a stepped arrangement. Preferred configurations of this stepped arrangement are designed and/or selected to facilitate gravity induced motile sperm selection. However, the sperm platform(s) may be configured in many shapes and sizes.

Preferably, the uppermost surface of the sperm platform and the single cell handling device are positioned relative to one another, generally, at a central focal plane. Co-location across a substantially common plane is preferred for the efficient collection of sperm and oocyte during the performance of an ICSI procedure. The sperm platform is preferably configured as a platform surface localised with respect to the single cell handling device to position and collect sperm.

The sperm platform itself may be configured in many shapes and sizes. Furthermore, each step of a stepped platform may be configured in many shapes and sizes. Any number of steps may be selected for the construction of the stepped platform.

Preferably, the cell handling array may comprise an unlimited number of single cell handling devices, each adapted to engage with the other. Preferably, the cell handling array will be a linear array in the horizontal plane, but alternatively may stack in the vertical plane or a mixture of both. Preferably, the single cell handling device will clip together in the horizontal plane and stack in the vertical plane or alternatively may slidably engage with one another in the horizontal or vertical plane.

In a preferred form, the cell culture base is adapted to connect with another cell culture base or cover. The base is preferably configured to physically stabilise the cell handling array, when placed upon a surface or when connected with another component or apparatus.

Preferably, base wells described herein are comprised in a culture system, further comprising a support platform and culture dish. Preferably, base wells described herein are formed within a culture dish and/or support platform.

Additional features may be provided in the culture systems described herein. For example, an identification label may be associated with each microwell to allow for the individual identification of the microwell and the contents contained therein. Such measures enable traceability while the culture system is in use, for instance, they may enable traceability of oocytes whilst performing ICSI procedures.

Preferred identification labels may provide numbering, lettering or other signs visible at the central focal plane of the spam platform and single cell handling device. This enables visibility of the identification label whilst performing an ICSI procedure.

Preferably, identification labels may be configured so as to be legible at different angles or orientations. For example, dual labelling may be provided for viewing different labels at 0° and 180°.

In further broad aspects, embodiments described herein relate to methods for performing intracytoplasmic sperm injection (ICSI) procedures.

Preferred methods for performing ICSI comprising the steps of;
Preparing a culture dish with media, a sperm sample and an oocyte sample,
Covering the culture dish with oil,
Separating the sperm sample and the oocyte sample,
Inserting an injection pipette into micromanipulator,
Immobilising a set of motile, morphologically normal sperm sample from the sperm sample,
Collecting the normal sperm sample in the injection pipette,
Carrying the collected normal sperm sample to the oocyte sample,
Selecting an oocyte, injecting the oocyte and aspirating a small volume of the oocyte cytoplasm into the injection pipette,
Injecting the oocyte cytoplasm and normal sperm sample into the selected oocyte,
Withdrawing the injection pipette.

In further broad aspects, embodiments described herein relate to methods for the manufacture of single cell handling device or cell handling arrays.

Preferably single cell handling devices or cell handling arrays described herein are manufactured as a two part construction comprising a two photon polymerised [2PP] printed part inserted into a polystyrene injection moulding (or alternative transparent and non-cytotoxic material).

Preferably, such methods comprise steps for the mechanical fitting of two parts. Such methods accommodate the mechanical fitting of uncured parts, followed by post curing assembly.

In an alternate construction, components maybe bonded, clip assembled, or a combination of both.

Methods for the manufacturer of cell culture systems described herein comprise the manufacture of a base well and a cover, having four sides and a top, and configured to fit over the uppermost surface of the base well.

Methods for the manufacture of the device are intended to support variations of the base well and cover, customisable to suit available off the shelf dish designs or custom designs of the base well and cover.

Methods for the manufacture of the device may permit the separation of component manufacture and final assembly.

Methods for the manufacture of the device may include features to support the automation of assembly and quality control inspection.

The term ‘cell’ as used herein is to be understood as interchangeable with the term ‘cellular material’ and shall refer to a cell, group of cells, tissue, or organoid which is the subject of the invention described herein.

As used herein the term ‘cell culture’ shall describe any tools or processes in which cellular material is isolated and maintained under controlled conditions for testing, growth, observation, experimentation, harvesting of the culture media, or other biological science processes.

As used herein the term ‘device’ shall describe fabrications which are produced at a micron scale, for example between 0.1 microns and 1000 microns. Devices shall encompass both static and mechanical devices as well as devices in the area of microfluidics.

As used herein the term ‘maintaining a cell’ shall refer to any process in which cellular material is stored in a controlled environment so as to produce the conditions required for viability. The term ‘culturing a cell’ shall refer to the processes of cell culture.

As used herein the term ‘cryopreservation’ or ‘associated cryopreservation’ shall refer to vitrification or freezing in an interchangeable fashion.

As used herein the term ‘handle’ and derivative terms such as ‘handling’, ‘handled’ etcetera are to be understood to comprise all forms of physical manipulation including, but not limited to, collecting, picking up, holding, grasping, gripping, nudging, rolling, touching and moving at the like.

As used herein the term ‘stabilize’ and derivative terms such as ‘stabilizing’, ‘stabilized’ etcetera are to be understood to comprise a physical state of holding an object relatively stable, firm, or steadfast wherein the term is relative to a state in which a stabilizing intervention is absent.

As used herein the term ‘moat’ is used to define a closed loop channel or trench that may receive and maintain fluid therein. The term ‘moat’ is not to be understood to imply any limitation of scale, and may indeed be formed at a microscale.

Broad embodiments of the invention now will be described with reference to the accompanying drawings together with the Examples and the preferred embodiments disclosed in the detailed description. The invention may be embodied in many different forms and should not be construed as limited to the embodiments described herein. These embodiments are provided by way of illustration only such that this disclosure will be thorough, complete and will convey the full scope and breadth of the invention.

Broad embodiments of the invention include the cell handling device for holding oocytes and optionally may include the cell handling device for holding sperm.

### DETAILED DESCRIPTION OF EMBODIMENTS

### BRIEF DESCRIPTION OF THE FIGURES

FIG. 1 provides a top view of a single cell handling device according to a raised microwell embodiment of the invention.

FIG. 2 provides a side perspective view of a single cell handling device according to a raised microwell embodiment of the invention.

FIG. 3 provides a side view of a single cell handling device according to a raised microwell embodiment of the invention.

FIG. 4 provides a cross-sectional view of a single cell handling device according to a raised microwell embodiment of the invention.

FIG. 5 provides a top view of a microwell array and sperm platforms of a single cell handling device according to a raised microwell embodiment of the invention.

FIG. 6 provides a side perspective view of a microwell array and sperm platforms of a single cell handling device according to a raised microwell embodiment of the invention.

FIG. 7 provides a top view of a sperm platform according to a raised microwell embodiment of the invention.

FIG. 8 provides a top transparent view of a microwell according to a raised microwell embodiment of the invention.

FIG. 9 provides a front cross-sectional view of sperm platform according to a raised microwell embodiment of the invention.

FIG. 10 provides a front cross-sectional view of microwell according to a raised microwell embodiment of the invention.

FIG. 11 provides a top view of a single cell handling device and device cover according to a raised microwell embodiment of the invention.

FIG. 12 provides a side perspective view of a single cell handling device and device cover according to a raised microwell embodiment of the invention.

FIG. 13 provides a side view of a single cell handling device according to a raised microwell embodiment of the invention.

FIG. 14 provides a side cross-sectional view of a single cell handling device and device cover according to a raised microwell embodiment of the invention.

FIG. 15 provides a top view of a device cover according to a raised microwell embodiment of the invention.

FIG. 16 provides a side perspective view of a device cover according to a raised microwell embodiment of the invention.

FIG. 17 provides a side view of a single cell handling device according to a raised microwell embodiment of the invention.

FIG. 18 provides a side cross-sectional view of a single cell handling device according to a raised microwell embodiment of the invention.

FIG. 19 provides a top view of a single cell handling device according to an embedded microwell embodiment of the invention.

FIG. 20 provides a top view of a microarray of a single cell handling device according to an embedded microwell embodiment of the invention.

FIG. 21 provides a side sectional view of a single cell handling device according to an embedded microwell embodiment of the invention from the microarray side of the base dish.

FIG. 22 provides a side sectional view of a single cell handling device according to an embedded microwell embodiment of the invention from the sperm platform side of the base dish.

FIG. 23 provides a side sectional view of microwell through opposite moat walls of a single cell handling device according to an embedded microwell embodiment of the invention.

FIG. 24 provides a top view of a microwell of a single cell handling device according to an embedded microwell embodiment of the invention.

FIG. 25 provides a side perspective view of a base dish and cover of a single cell handling device according to an embedded microwell embodiment of the invention.

FIG. 26 provides a side perspective view of two stacked base dishes of a single cell handling device according to an embedded microwell embodiment of the invention.

FIG. 27 provides a side view of a base dish of a single cell handling device according to an embedded microwell embodiment of the invention.

FIG. 28 provides a side perspective view of a cover of a single cell handling device according to an embedded microwell embodiment of the invention.

FIG. 29 provides comparative studies assessing setup efficiency between conventional ICSI and an embedded microwell embodiment of the invention including a comparison of (a) equipment handling, (b) pipette setup, (c) oocyte handling, and (d) oocyte unloading time.

FIG. 30 provides comparative studies assessing procedural efficiency between conventional ICSI and an embedded microwell embodiment of the invention including a comparison of (a) oocyte injection handling, (b) injection time, (c) polar body orientation, and (d) injection time.

FIG. 31 provides comparative studies assessing the number of hand adjustments during oocyte injection between conventional ICSI and an embedded microwell embodiment of the invention.

FIG. 32 provides comparative studies assessing the level of invagination during oocyte injection between conventional ICSI and an embedded microwell embodiment of the invention.

FIG. 33 provides comparative studies assessing the developmental rate between conventional ICSI and an embedded microwell embodiment of the invention including a comparison of (a) fragmentation rate, (b) survival rate, (c) cleavage rates, and (d) blastocyst rates.

FIG. 34 provides comparative studies with human oocytes between conventional ICSI and an embedded microwell embodiment of the invention including a comparison of (a) injection rate, and (b) number of hand adjustments.

Several embodiments of the invention are described in the following examples.

### EXAMPLES

The examples relate to a two photon polymerization (2PP) 3D printing fabrication of a single cell handling device designed to adopt a raised microwell design or an embedded microwell design. The fabrication approach has been enhanced by creating a single piece printed device to hold up to twenty oocytes at a time. The functional and work-flow differences between the new ICSI techniques adopted for the single cell handling device (microICSI) and conventional ICSI (C-ICSI) were compared, assessing both procedural efficiency and embryo developmental outcomes. Finally, a pilot study is described which demonstrates the utility of the device for human oocyte microinjection.

The single cell handling devices described herein are suitable for holding oocytes and sperm during ICSI procedures. The exemplified devices comprise a base dish and a lid or cover. The base dish combines the functionality of a conventional ICSI cell culture dish with improved features for positioning the oocyte to eliminate the need for a holding pipette. The oocyte can be positioned without the need for vacuum manipulation of the cell. The configuration of the device is optimised to provide maximum support to the oocyte; to minimise deformation and invagination of the oocyte consequently improving the quality of the resultant zygote and/or increasing fertilisation rates.

Positioning oocytes in a fixed position within the dish eliminates the need for the vacuum pipettes and allows the embryologist to easily locate oocytes to perform ICSI, improving the efficiency in performing ICSI.

### General Base Dish and Microwell Design

The exemplified base dishes are designed to support oocytes during the ICSI procedure within a hemispherical structure (or cup) to remove the need for a holding pipette and suction. The oocyte is cupped during the microinjection within the hemisphere surrounded by a microwell. A channel allows the injection pipette to maneuver oocytes into position within the hemisphere or cup prior to sperm pickup where the oocyte can be handled and stabilized for microinjection.

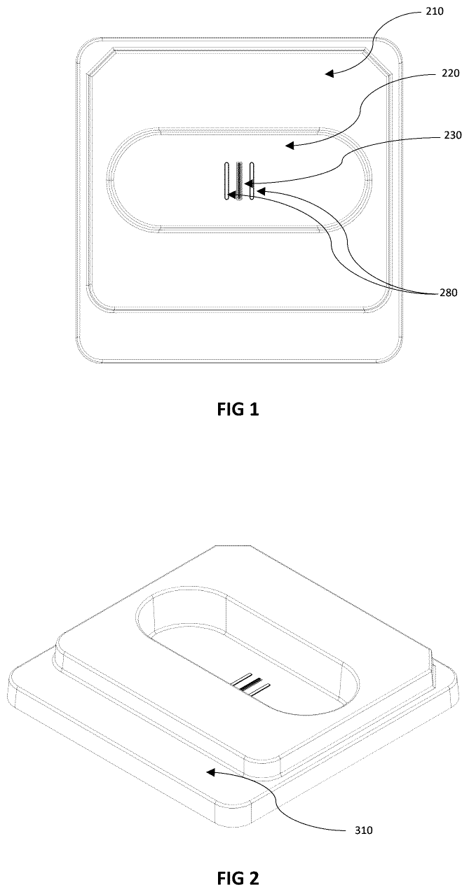

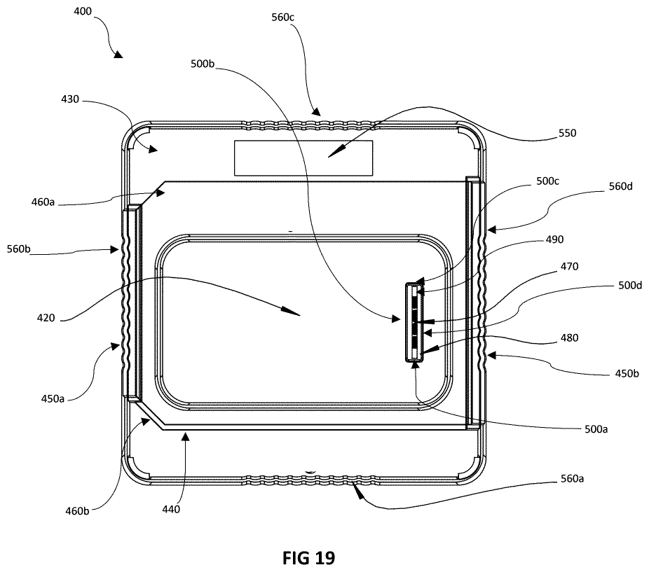

FIGS. 1 to 18 illustrate a raised microwell design in which the microwell array is fabricated in a raised fashion upon a base platform. Whereas, FIGS. 19 to 28 illustrate a variation to the fabrication method based on an embedded microwell design.

### Raised Microwell Design

With reference to FIG. 1 to 18, FIG. 1 shows a top view of a device base dish 210 having a single cell handling device located thereon according to embodiments of the invention. The base dish, 210, is comprised of a 65 millimeter square dish with provisioning for patient labelling. Base dish, 210, is located within a base well, 220. Base well 220 allows for media to be dispensed within base dish 210 and overlaid with oil.

Centrally positioned within base well 220, is microwell array 230. Microarray, 230, is comprised of multiple individual wells providing individual positions for holding and maintaining individual oocytes from a single patient therein. Sperm platforms, 280, are located adjacent at either side of the microwell array.

FIG. 2 shows a side perspective view of base dish 210. Base dish 210 is located above support platform 310 and is stepped up from support platform 310. Base dish 210 and support platform 310 are each approximately 4.5 millimetres high. The depth of base well 220 spans the height of support platform 310 and base dish 210, providing a depth for base well 220 of approximately 8.5 millimetres.

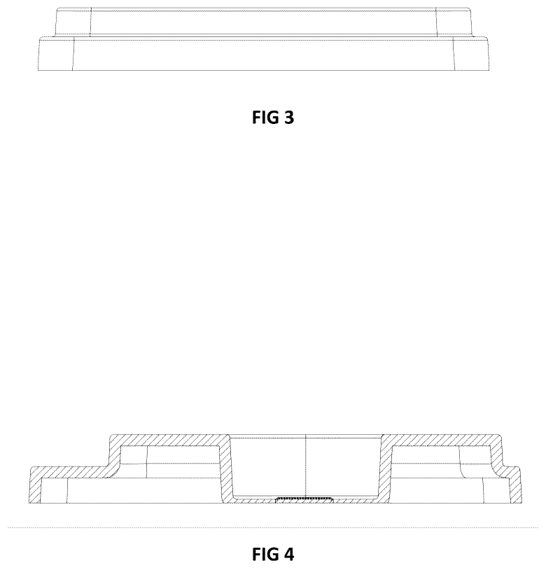

FIG. 3 provides a side view of the single cell handling device showing the stepping up between support platform 310 and base dish 210, which are approximately of equal height.

FIG. 4 provides a side sectional view of the single cell handling device; sectioned midway through base well, 220, and longitudinally through microwell array 230. As shown in FIG. 4, base dish 210 is offset above support platform 310. FIG. 4 shows the stepped profile of the device at the edge of the microwell array. FIG. 4 shows the depth of base well 220, which spans the height of the base dish, 210, and support platform 310. The side walls of base well, 220, are at a slope to facilitate injection moulding manufacture.

In use, base well 210 is filled with cell culture medium to immerse microwell array, 230, within the culture medium. The total capacity of the base well 210 is approximately 7.8 millilitres to allow for oil overlay.

A detailed view of microwell array 230 and sperm platforms 280 are provided at FIG. 5. A series of microwells, 240, are positioned lengthwise in a row to provide microwell array, 230. Each well provides an individual single cell handling device for holding and manipulating a single oocyte within each well. Each microwell array, 230, contains a series of oocytes from a single patient. The array is compact and linear for efficient visualisation, repositioning, and manipulation by the embryologist.

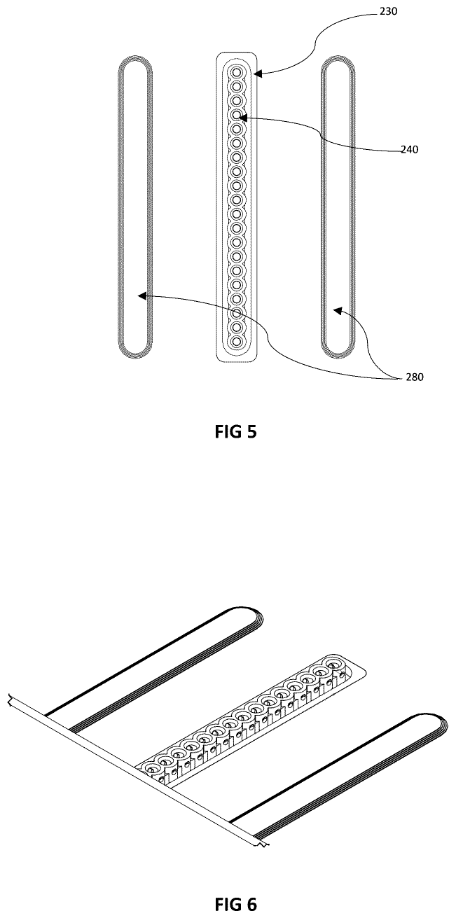

FIG. 6 shows a side perspective view of microwell array 230 and sperm platforms 280. Each individual microwell 240 within a microwell array 230 is printed to its adjacent well to form the microwell array 230 as a single unitary structure. A chamfer is provided at each microwell opening to enable the positioning of the embryologist's injection pipette for placement of an oocyte within the microwell, 240, and for the retrieval of a zygote formed therein.

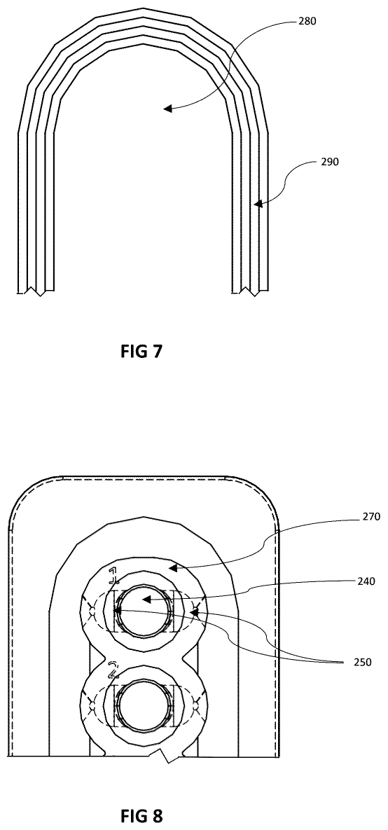

FIG. 7 shows a partial top view of sperm platform 280. Two sperm platforms 280 are located adjacent to either side of the microwell array 230. Sperm platforms 280 are located at the same height as the central plane of the oocytes. Alignment of the sperm platform and the oocyte microwell allows for sperm pick up, oocyte positioning and manipulation to perform an ICSI procedure within the same nominal focal plane. Thereby, minimal adjustment is needed by the embryologist to ensure that visualisation of the procedure is in focus.

Sperm platform 280 is positioned approximately 2 millimetres from the microwell array 230. This separates sperm from the oocytes to avoid contact that could result in uncontrolled fertilisation (TVF).

By separating the sperm platforms 280 from the microwell array 230, this also provides clearance for any angular positioning of the ICSI pipette when the embryologist is positioning the oocyte and performing ICSI.

Sperm platform 280 extends the full length of microwell array 230. This enables sperm pick up to be performed by the embryologist in close proximity to the oocyte. This minimises handling, for the efficient completion of an ICSI procedure by the embryologist.

By providing two sperm platforms 280 either side of the microwell array 230, the embryologist is able to perform an ICSI procedure according to their preference for either left handed or right handed manipulation of the ICSI pipette.

Sperm platform 280 comprises a series a vertical sperm platform steps, 290. Sperm platform steps 290 enable gravity induced motile sperm selection by the embryologist. In such procedures, motile sperm can be located at the highest step of sperm platform steps 290, whereas dead and immotile sperm remain at the lowest point of the dish.

Turning to FIG. 8, adjacent to microwells 240, at either side, are two opposing microwell hemispheres, 250. Each microwell hemisphere 250 has formed therein a hemisphere recess extending circumferentially around the perimeter of the microwell wall; such that each microwell 240 has formed within its walls two opposing hemisphere recesses.

The oocyte is positioned within the recess, which provides optimal support of the oocyte during an ICSI procedure.

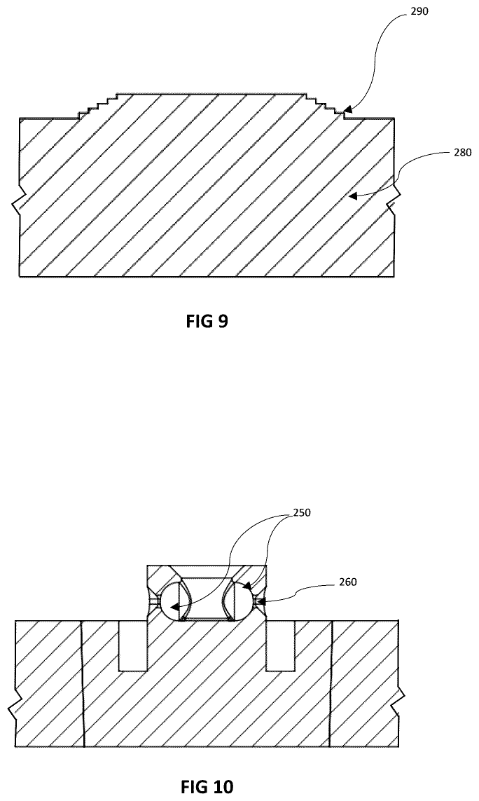

With reference to FIG. 10, the hemispheres 250 are positioned tangentially at the base of the microwell 240. This allows oocytes that have been placed on the base of the microwell to be readily moved by the ICSI pipette (via the access channel 260) to the opening of the opposing hemisphere before being positioned into the full depth of the hemisphere cup.

The hemispheres 250 are formed as a recess within the microwell 240 wall thickness. Oocytes positioned at the opening are partially supported on their circumference, allowing the oocyte to be orientated with the ICSI pipette (via the access channel 260) prior to full insertion into the hemisphere cup where the oocyte is fully supported and prevented from rolling.

During a ICSI procedure, the oocyte is positioned within one of the two possible hemisphere recesses. The embryologist may select which of the two hemisphere recesses they prefer, to allow for their preference of left handed or right handed manipulation. The embryologist is able to access the oocyte with an injection pipette through access channel 260. The oocyte is supported within the hemisphere recess during the ICSI procedure, minimising invagination of ooplasm during injection and direct deposition of the sperm within the ooplasm, without the need for cytoplasm withdrawal into the injection pipette. Similarly, microwell hemispheres 250 provide support to the oocyte during withdrawal of the injection pipette. Microwell hemispheres 250 are approximately between 100 microns and 200 microns in diameter to support the expected ranges of oocyte sizes including the zona pellucida.

With reference to FIG. 9, which provides a front cross sectional view of sperm platform 280, sperm platform steps 290 are clearly shown. Sperm platform steps 290 allow motile sperm to be located at the highest step of sperm platform steps 290, whereas dead and immotile sperm remain at the lowest point.

FIG. 10 shows a front cross sectional view of microwell 240. Each microwell 240 comprises two opposing microwell hemispheres 250 and two opposing access channels 260. Access channels 240 allow the embryologist to insert an injection pipette laterally into the well to perform an ICSI procedure. The channel is sized as small as possible to maximise the surface contact of the hemisphere for optimal support of the oocyte. Access channels 240 have a chamfered opening formed therein to allow the injection pipette access to microwell 240 and microwell hemispheres 250 and allow the micropipette to be angled, in order to adjust for the positioning of the oocyte prior to injection.

While opposing access channels 260 primarily provide the embryologist with the option for their preference of left handed or right handed manipulation, this symmetry serves additional functions. The second access channel facilitates the clearing of bubbles when microwell 240 is filled with media, additionally it prevents the build up of back pressure behind the oocyte when the embryologist is performing an ICSI procedure.

Identification of microwell arrays 230 and individual microwells 240 is provided by localised over-polymerisation during 2PP printing of the insert. This allows numbering to be created by changing the material refractive index. It is positioned at the central plane of the oocytes to allow for identification by the embryologist whilst performing an ICSI procedure.

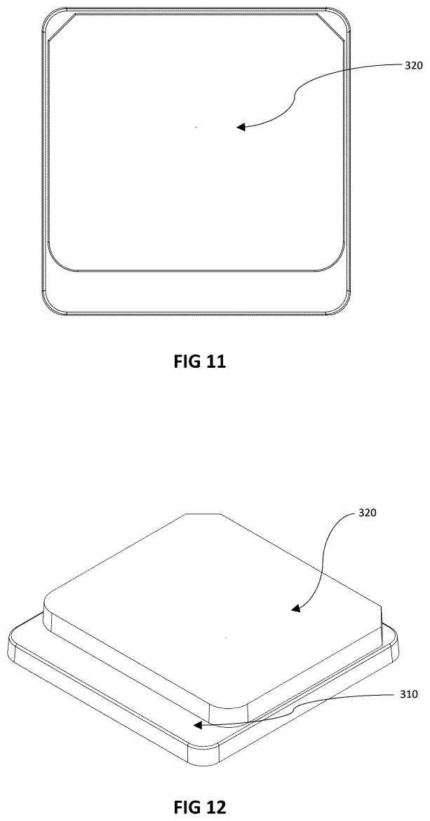

FIG. 11 provides a top view of base dish cover 320, which is shaped to cover the entirety of base dish 210 and remain supported upon the top surface of support platform 310. FIG. 12 provides a side perspective view of base dish cover 320 covering base dish 210 showing the stepping up of base dish cover 320 on support platform 310.

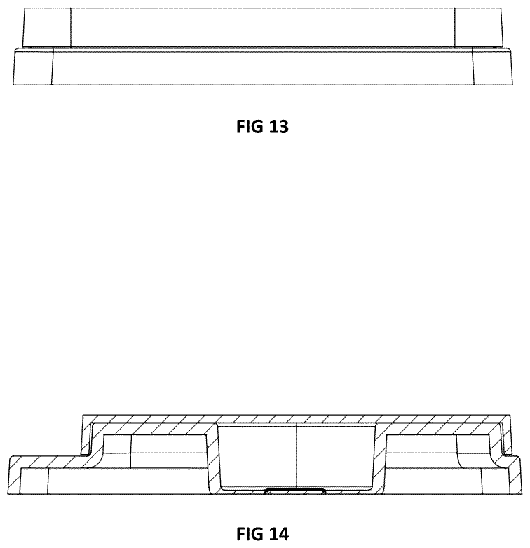

FIG. 13 provides a side view of base dish cover 320 and support platform 310. Side sectional view is provided at FIG. 14, showing the fit of base dish cover 320 over base dish 210 to form a tight fit thereon.

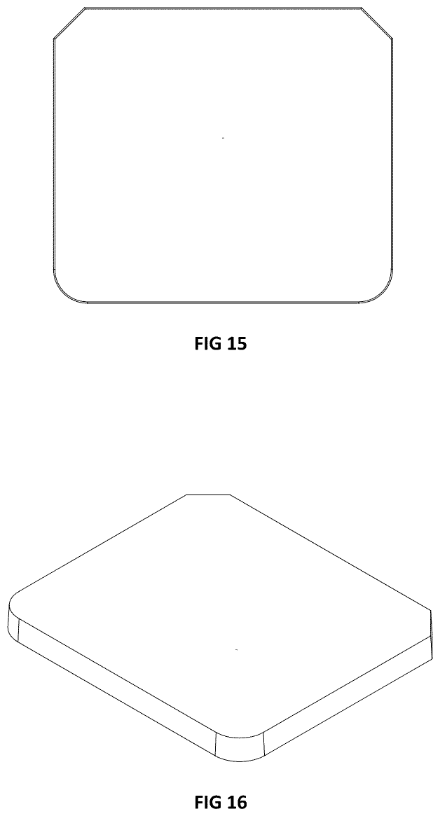

Top view of base dish cover 320, provided at FIG. 15, and side perspective view of base dish cover 310, provided at FIG. 16, show the shape of base dish cover 320. Two rounded corners are provided in base dish cover 320 and are opposed with two angled corners formed into base dish cover 320.

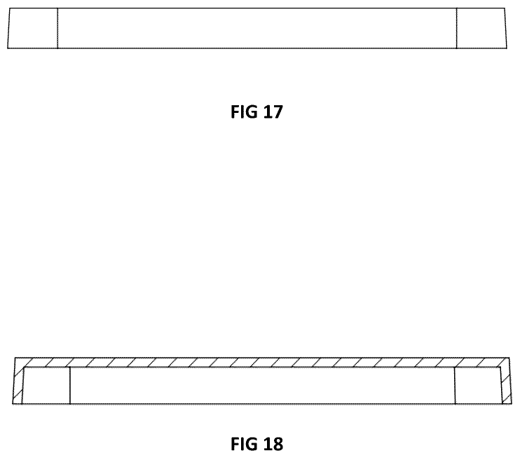

FIGS. 17 and 18 similarly illustrate the shape of base dish cover 320; from a side view at FIG. 17, and from a side sectional view at FIG. 18.

### Embedded Microwell Design

Turning to FIGS. 19 to 28, FIG. 19 shows a top view of a device base dish 400, having embedded microwells located therein. As illustrated in FIG. 19, the embedded microwell design includes asymmetric placement of the microwell array 470 to allow adequate functional space for the sperm preparation area 420. The embedded microwell design is simplified by providing a single planar sperm preparation area 420 (in place of the oval channelled sperm platform of the raised design) embedded within raised platform surface 430. Cover recess 440 provides physical guidance for the placement of the cover. Two opposing parallel sides of cover recess 440 extend through raised platform surface 430 and through two opposite base dish walls 450a and 450b. Cover recess 440 is angled at two recess corners 460a and 460b to provide guidance for accurate fitment of the cover.

Microwell array 470 is provided within the perimeter of a recessed moat 480 formed within the raised platform surface 430. The moat 480 comprises a base 490 and a wall 500 having four sides, 500a, 500b, 500c and 500d; all formed by a space fabricated within base dish 400. The base 490 and sides of moat 480 are designed to contain a large volume of microwell media for bathing the cellular contents of the microwells in a volume sufficient to dilute any byproducts of cellular activity from the cells contained within the microarray.

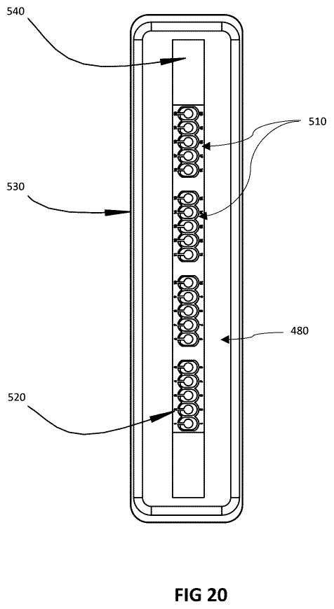

Microwell array 470 comprises spaced microwell groupings 510 of five microwells 520 in each grouping as depicted in FIG. 20. This configuration discourages bubble formation from media contained in in the moat 480. Moat 480 is defined by a multistage perimeter 530 to promote seamless transition of the needle across the dish to each microwell 520. Extended ends 540 provide a point of reference to allow ease of alignment and assembly. Label area 550 is provided on base dish to allow custom identification of the base dish. Partially knurled edges 560a, 560b, 560c, and 560c are provided on four sides of the base dish to allow for the ease of handling the dish during transport.

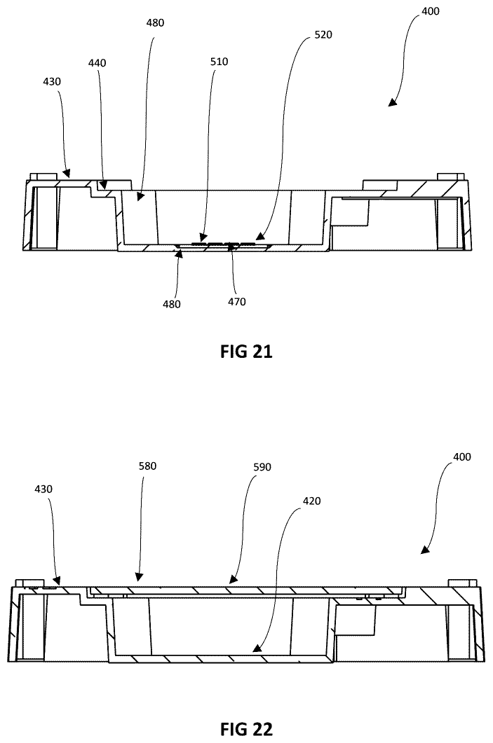

FIGS. 21 and 22 provide sectional side views of the embedded single cell handling device from the microarray side (FIG. 21) and from the sperm platform side (FIG. 22). FIG. 21 illustrates the embedded design of microwells 520, microwell groupings 510, microwell array 470, and moat 480. The embedded fabrication is achieved through fabrication of base dish 400 such that raised platform surface 430 steps down at approximately 93° to form cover recess 440 shaped as a ledge to maintain cover (not shown) thereon at the same plane as raised platform surface 430. A further step down forms moat 480 contiguous with the cover recess 440 and raised platform surface 430 fabricated within base dish 400 and having a wall formed at 90° from cover recess 440.

The view provided at FIG. 22 shows the sperm preparation area 420 fabricated in the same plane as microwells 520. Cover 580 occupies the space above cover recess 440 and forms a cover upper surface 590 in the same plane as raised platform surface 430.

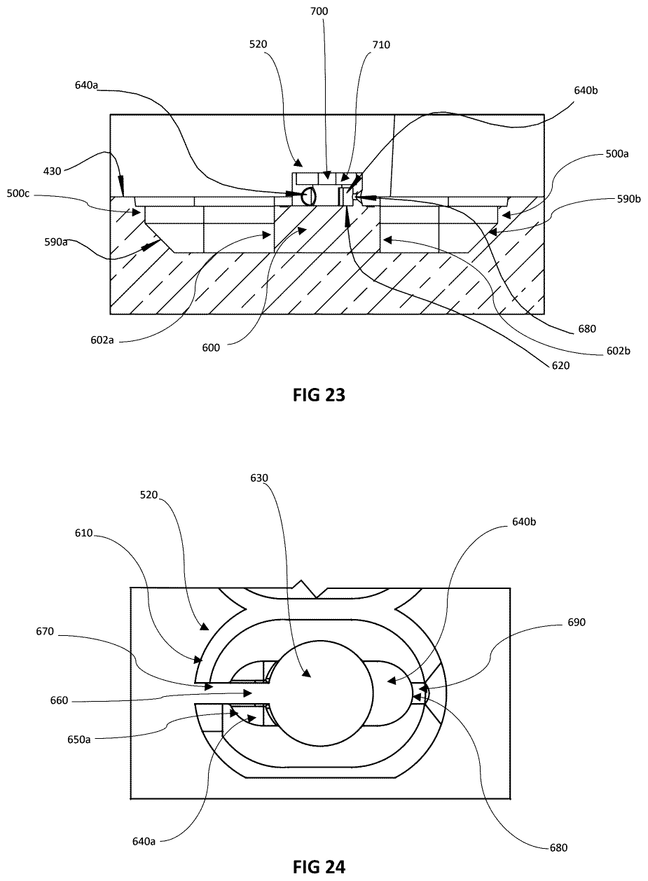

FIG. 23 provides a side sectional view of microwell 520 through moat walls 500a and 500c. The walls are fabricated to add chamfers 590a and 590b to the base of the moat 480. This acts as a visual cue for the user while the dish in use to indicate that a user's needle has entered the moat area. Moat 480 is recessed into the dish to ensure the raised platform surface 430 is aligned with the focal place of the microwell. Base dish 400 comprises a microwell support portion 600 located beneath each microwell 520 along the length of the microwell array 470 and positioned entirely within the perimeter of moat 480. Edges 602a and 602b of the microwell support portion 600 allow the base dish 400 to have good thermal contact with the heated stage (not shown) typically used during ISCI procedures.

The configuration of microwell 520 is illustrated in greater detail in FIG. 24. Each microwell is formed by a single curved exterior wall 610 and a flattened microwell base 620. Each microwell is open from above to provide access to the microwell for larger objects including oocytes, or pipettes for transferring fluids directly to the microwell. A rounded well cavity 630 is provided centrally within the microwell and is flanked at opposite sides by two rounded oocyte holding cups 640a and 640b. The diameter of well cavity 630 is approximately 250 microns, to support the expected ranges of oocyte sizes of 100 microns to 200 microns including the zona pellucida.

A wall forming well cavity 630 comprises two openings to form a contiguous shape with the walls forming the two oocyte holding cups. Inner holding cup 640a is oriented proximally to the sperm preparation area and is formed by an inner holding cup wall 650a having a channel opening 660 extending through the inner holding cup wall 650a to injector channel 670. Injector channel 670 is formed by channel wall 680, formed through the single curved exterior wall 610 of the microwell 520 to permit access to the microwell for smaller objects such as microinjection apparatus. The shape of channel wall 680 provides a guideline for the placement of a microinjection needle to align with the centre of the outer holding cup 640b. It is also shaped to allow the user to orient the polar body of the oocyte using the microinjector prior to an ICSI procedure.

Outer holding cup 640b is located distally to the sperm preparation area. Outer holding cup wall 650b is formed in symmetry with the inner holding cup wall 650a, both taking the general shape of a half hemispherical cylinder. Outer holding cup wall 650b has an exhaust opening 680 formed therethrough. Exhaust port 690 is provided at the exhaust opening 680 to ensure that the oocyte can be placed in the cavity formed within the outer holding cup 640b without fluidic resistance impacting the oocyte. Whereas the cavity formed within inner holding cup 640a maintains the oocyte therein to eases removal of a microinjector from an oocyte following microinjection.

The inner surfaces of microwell 520 have been designed to enable high quality visualisation. A flattened shape for the microwell base 620 has been adopted to promote visualisation of oocyte during a microinjection procedure. A half cylindrical cell stabilizing protrusion has been adopted for the outer holding cup 640b to promote visualisation. A flat visualisation lens 700 extends across the top opening 710 of microwell array 520 to allow visualisation of microwell identification information during use. Embossed numbers in cutout slots on each microwell array (not shown) allow for the identification and traceability of samples.

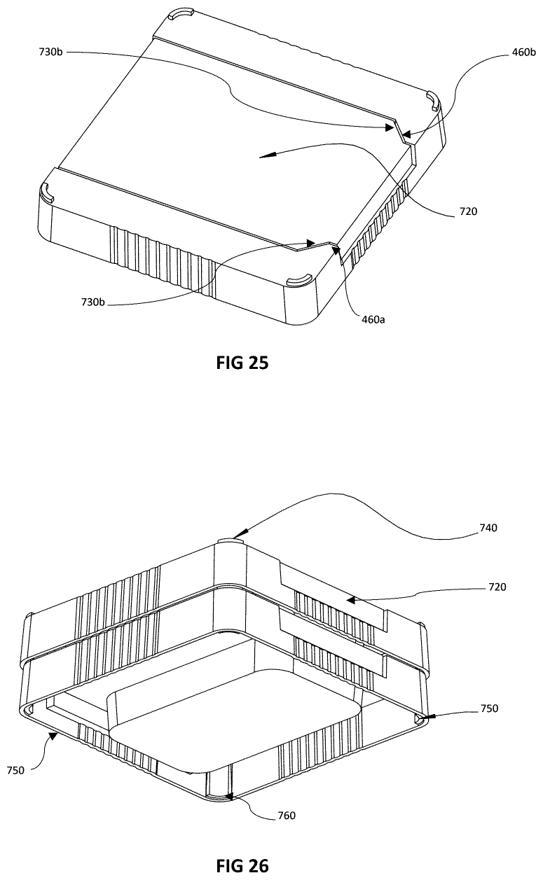

FIGS. 25, 26, 27 and 28 illustrate the exterior of base dish 400 and cover 720. FIG. 25 illustrates the fitment of cover 720 upon the cover recess 440 formed within the raised platform surface 430 and through two opposite base dish walls 450a and 450b. Cover 720 comprises two angled cut outs 730a and 730b at either corner of cover 720. These correspond with two recess corners 460a and 460b to provide for accurate fitment of the cover.

FIG. 26 illustrates the capacity to stack multiple base dishes with each base dish has an individual cover 720 fitted thereto. Upper stacking lugs 740 are located at each corner of the raised platform surface 430 to provide fitment with the underside surface 750 of the base dish. Under stacking recesses 760 are provided within the underside surface 750 of the base dish to correspond with the shape of the upper stacking lugs 740 to aid the secure stacking of multiple base dishes.

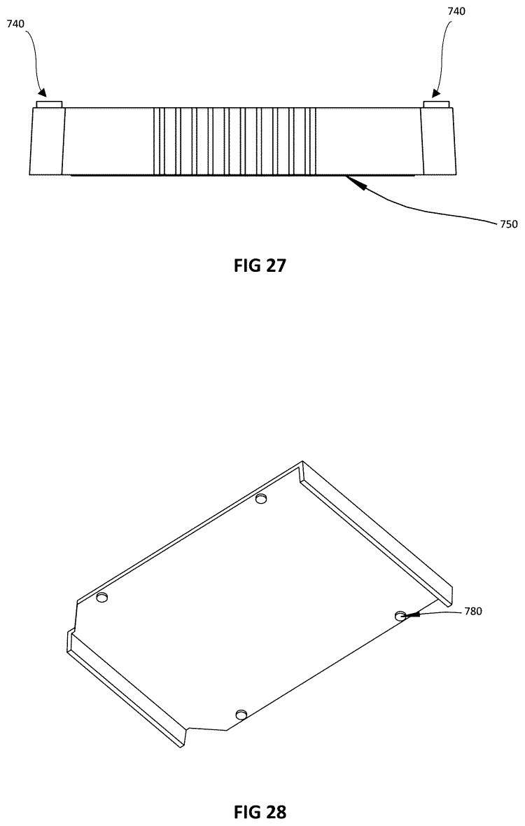

FIG. 27 illustrates the underside surface 750 of the base dish which is shaped to promote thermal contact with a heated stage during an ICSI procedure. FIG. 28 shows spacer pins 780 located at an underside of cover 720 to allow gas exchange during incubation.

### Fabrication of Devices

The microarrays were printed with a NanoOne 1000 high-resolution 2PP 3D printer and a proprietary acrylate-based 2PP resin (‘UpFlow’, UpNano GmbH, Vienna, Austria). Following modelling of 3D parts in CAD software (‘Solidworks’, Dassault Systemes Solidworks Corp., Waltham, Massachusetts, USA), exported STL files were imported into Think3D software (version 1.7.3, UpNano GmbH). Parts were printed using a 10× objective operating in coarse mode, or “voxel slice mode,” with a layer thickness of 5 microns, line distance of 4 microns and power setting of 430 mW.

The microarrays were post processed by washing in baths of propylene glycol monomethyl ether acetate (PGMEA≥99.5%) for 3×10 mins and 2-propanol (99.9%) for 1×3 min and 1×2 min before removal from the print substrate with a razorblade and subsequent air drying. Dried parts were cured for 5 mins in an ODS Cure Box (One Digital System, Incheon, Korea) at 60% power before plasma treatment. Microarrays were treated with an oxygen plasma at a pressure of 780-820 mTorr for 60 sec using a PDC-002-HP chamber (Harrick Plasma, NY, USA). Plasma treated arrays were adhered to commercial polystyrene dishes (Product No: 16006, Vitrolife Pty. Ltd., Sydney, Australia) using a small amount (~25-30 123 mg) of UpFlow resin followed by 2×5 min curing treatments in the ODS Cure Box.

After curing, dishes were washed twice in 3 mL of 96% ethanol (Chem-Supply Pty Ltd, 125 Gillman, SA, Australia) and rinsed twice in filtered (pore size 0.22 micron; Millipore, Bedford, MA, USA) 5% 7× detergent (MP Biomedicals Australasia, Seven Hills, NSW, Australia). Dishes were then rinsed three times in Milli-Q water to remove residual detergent and left to dry on a heat plate set to 37.5 °C overnight (Ratek Instruments Pty Ltd, VIC, Australia). At each step of washing, a P1000 micropipette was used to pipette washing solution over the microarray, thereby flushing the wells.

For human oocytes, an adjustment to the size of the hemispheres was performed to accommodate the larger size of oocytes prior to printing the microarrays. Thereafter, printing, post-processing and washing were conducted as described above.

Toxicity testing of previous 2PP parts were shown to be non-toxic (Yagoub S. H. et al. (2022), Yagoub S. H. et al. (2014) and McLennan H. J. et al. (2023)) using a mouse embryo assay (MEA) that included both negative and positive controls (IVF VET Solutions, SA, Australia). This was repeated in the current study with no toxicity detected. Cover fabrication followed the same parameters and washing protocol detailed above.

### Method for Use of Single Cell Handling Device

The following methods may be readily adapted for use of either the embedded or raised microarray designs.

The base dish is prepared by labelling the base dish and obtaining PVP, media oil, p10 and p20 pipettes, pipette tips, a serological pipette and a serological pipette, and working under a laminar flow hood.

Approximately 10 microlitres PVP is added from 2PP part in a long strip parallel to the microarray on the available side. 30 microlitres of culture media is added to the moat on the injection slot side of the microarray.

The base dish is orientated with the injector slot marker facing the user's preferred injector side. The lid is then removed. 10 microlitres of PVP is added to the base dish with a p10 pipette; approximately 2 mm from the 2PP microarray. The PVP drop is stretched along the length of the microwell array. 20 microlitres of media is slowly added to the injector side of the moat with a p20 pipette. This step is repeated until a total of 30 microlitres of media is added to the dish. The media should be spread around the entire moat of the microwell array.

6 mL of oil is placed on a section of the dish clear from the PVP and the microwell array. The lid is placed back on the dish. The dish is then equilibrated by placing the dish in an incubator.

Cryopreserved sperm is warmed and then prepared by removal of seminal fluids and other preservatives via centrifugation (washing). Washed sperm are loaded onto the base dish using a 300 microlitres handling pipette, the equilibrated dish, and a dissecting microscope with a heated stage and/or within a heated crib. Work is performed under a dissecting microscope with heated stage and/or within a heated crib. The sperm sample is gently agitated by inverting to complete the dispersal of sperm evenly in solution. A sperm aliquot is aseptically collected using the 300 microlitres handling pipette. The sperm sample is then slowly dispensed onto base dish PVP strip by depressing the handling pipette equally along the PVP stretch, so the sperm are evenly distributed.

The oocytes are loaded into the microwells of the base dish by obtaining the post denuded culture dish with oocytes. Under a dissecting microscope with a heated stage and/or within a heated crib with magnification set at 3× magnification to see five wells at a time. Surface bubbles must initially be removed before collecting the oocytes. Five oocytes are aspirated using the handling pipette and are moved to the base dish such that a single oocyte is deposited into each microwell. This step is repeated until all oocytes are loaded into the microwells. The base dish loaded with oocytes may then be moved to the microscope stage for performing ICSI.

If required, mock injections may be undertaken at this stage with either debris or blue beads. An injection pipette is obtained and the injector is angled slightly for immobilising sperm. The injector is set to position 1 (if workstation allows) by focusing the injector with the sperm and the meniscus of the PVP drop. The injector is raised slightly and the stage is manoeuvred to the microwell and focussed on a microwell and egg. The injector is brought into same plane of focus as egg and placed in parallel with the injection slot of the microwell. The injector is pushed to glide through the injection slot and to manoeuvre the oocyte gently into the hemisphere, with the polar body orientated correctly. The injector is pulled back to glide toward the injector slot, while keeping it parallel with slot. The injector is gently slid out of the oocyte then nudged back into microwell with the injector tip (to aid unloading later).

The stage is moved to shift the focal point to the next microwell. All polar bodies are orientated throughout all microwells. The injector must be positioned over the PVP strip and changed to position 1 or adjusted to be in line with sperm. A single sperm is selected, immobilised and gently aspirated. After raising the injector, the microscope stage is moved to focus on the middle of the oocyte in the first microwell. The sperm is injected into the oocyte. These steps are repeated until all oocytes are injected.

By way of variation, the injection pipette may be loaded with more than one sperm (nominally around three) and a serial injection sequence can occur, which further reduces time. This “sequential injection” process is known in the art as “shotgun injection”.

The arm of the injector is raised to give clearance for the dish to be removed. The dish is removed to the dissecting microscope for unloading post procedure oocytes. The oocyte handling pipette tip is rinsed in the wash dish. The five post procedure oocytes are aspirated from the base dish by holding the pipette at 80°. These are pipetted around the dish until washed. The oocytes are aspirated and placed in a culture dish. These steps are repeated for each of the oocytes.

### Results of Validation Tests and Pilot Studies

### Oocyte Collection and Maturation

Porcine ovaries collected from a local abattoir from cycling gilts and sows were transported back to the laboratory in phosphate buffered saline (PBS) in thermos between 30-35 °C. Ovaries were washed in warm saline and placed into beakers within a water bath set to 37 °C. Clear antral follicles between 3-6 mm were aspirated with a 21-gauge needle with constant suction (1 L/minute) into 9 mL Greiner No Additive Vacuette tubes (Interpath Services, Somerton, VIC, Australia). Cumulus-oocyte complexes (COCs) with several layers of cumulus cells were isolated from the aspirant. Once pooled together, COCs were washed 3 times and placed into groups of 50 in 500 mL of IVM medium consisting of Medium (Gibco, Thermo Fisher Scientific, Waltham, MA, USA) supplemented with 10 IU/mL equine chorionic gonadotropin (Folligon MSD, Australia), 10 IU/mL human chorionic gonadotropin (hCG, Chorulon MSD, Australia), 5 mg/mL insulin, 10 ng/mL Epidermal Growth Factor (EGF), 1 mM cysteamine, 100 mg/ml Na-pyruvate, 75 mg/mL Penicillin-G, 50 mg/mL Streptomycin sulphate, and 10% filtered sow follicular fluid under mineral oil (Origio, Sydney, NSW, Australia) in a 4-well NUNC dish (Thermo Fisher). Oocytes were matured for approximately 40 hours in a humidified atmosphere of 5% CO₂ in air at 38.5 °C.

At the end of the maturation period, morphologically normal COCs were isolated using a small-bore glass pipette (Rowe Scientific, Lonsdale, SA, Australia). Groups of 50-60 COCs were placed into separate 5 mL round bottom tubes with IVM medium without any hormones for transportation at 37 °C in a transport incubator (CryoLogic, Blackburn, VIC, Australia) until time of denudation, approximately 1 hour after cessation of maturation.

### Intracytoplasmic Sperm Injection (ICSI)

For C-ICSI, 5 microlitre drops of 7% polyvinylpyrrolidone (PVP; Origio Australasia Pty Ltd, CooperSurgical, Denmark) for priming the injection pipette and seven drops of 5 microlitres of 4-(2-hydroxyethyl)-1-piperazineethanesulfonic acid (HEPES) buffered NCSU23 (ART Lab Solutions Pty Ltd, Adelaide, SA, Australia) were placed into a Vitrolife ICSI dish in a 3×3 droplet grid. 5 microlitres of HEPES-buffered NCSU23 was added to the central PVP drop to make a 3.5% PVP solution for sperm to partially slow motility. The dish containing the microarray differed in medium arrangement, comprising two strips of 5 microlitres of 7% PVP laid down the left side of the microarray, with 5 microlitres of HEPES-buffered NCSU23 added to the PVP droplet closest to the array for sperm pick up. 20 microlitres of HEPES-buffered NCSU23 was pipetted over and around the microarray moat. All dishes were covered with 5 millilitres of MEA tested paraffin oil (Merck Group, Macquarie Park, NSW, Australia) and heat equilibrated to 38.5 °C for a minimum of 3 hours.

Freshly collected and extended semen was purchased from a local boar stud (Sabor, Clare, SA, Australia). Semen was equilibrated to room temperature from storage temperature (18 °C) and was centrifuged at 300 g for five minutes. The supernatant was discarded, and the remaining pellet was resuspended in 10 mL of warm sperm wash media (Medium 199 supplemented with 0.1 mg/mL sodium pyruvate, 0.9 mg/mL calcium-lactate, 0.075 mg/mL penicillin G, 0.05 mg/mL streptomycin sulphate, and 10% fetal bovine serum (FBS)). The resuspended pellet was then centrifuged and further resuspended in sperm wash medium. Sperm were further diluted with warm HEPES-buffered NCSU23 to obtain a final concentration of ~5×106 sperm/mL.

Mature COCs were treated with 0.1 mg/mL hyaluronidase in HEPES-182 buffered NCSU23 for one minute and gently pipetted using a 130-140 micron internal diameter (ID) Flexipet pipette tip and Cook Adjustable Handle (Cook Medical Pty Ltd, Eight Mile Plains, QLD, Australia) to remove cumulus cells. Oocytes were washed three times in HEPES-buffered NCSU23 post denuding and assessed for suitability and the presence of a single polar body for selection to undergo the ICSI procedure.

Pipette setup was timed for the conventional ICSI and the microarray procedure, as only one injection pipette is required for microarray as opposed to a holding pipette and injection pipette for conventional ICSI. This included the time taken for focussing on the medium drop and pipettes during pipette positioning in media under oil. Two ICSI workstations were utilized to allow operators to conduct experimentation concurrently and avoid oocyte aging. A Nikon ICSI station utilized a Nikon TE2000 Eclipse inverted microscope (Nikon Corporation, Tokyo, Japan) combined with Eppendorf micromanipulators (Eppendorf, Hamburg, Germany) and air microinjector (Eppendorf) for the 25 micron ID holding pipette and oil microinjector (Eppendorf) for the 6 micron ID injection pipette, both sourced from ICSION (Thebarton, SA, Australia). A second Olympus station comprised of an Olympus TX83 inverted microscope (Evident Corporation, Tokyo, Japan) and Narishige ON4 micromanipulators (Tokyo, Japan), with Narishige injectors (IN-21 for oil, IN-9B for air). Treatment order per operator was randomized for each replicate. Operators used a combination of 10× objective with 2× magnifier on the Olympus microscope and 20× objective on the Nikon microscope to visualise sperm and perform the ICSI process.

For conventional ICSI, within a custom-built modified heated infant crib set to 38.5 °C, 10-20 matured oocytes were transferred into the 5 microlitre HEPES media droplets using a 170 micron flexi-pipette just prior to the ICSI procedure. Approximately 3 microlitre of diluted sperm was pipetted into the 3.5% PVP droplet using a flexi-pipette. The dish was carefully transferred to either inverted microscope onto a heated stage set to 38.5 °C. For each injection, a single motile sperm with good morphology was selected and immobilized within the 3.5% PVP sperm droplet. An oocyte was held in place with the holding pipette, with the polar body rotated to either the 6 or 12 o'clock position using the injection pipette. The injection pipette with the sperm located at the tip was introduced into the oocyte at the 9 o'clock position, with the ooplasm aspirated into it to ensure the oolemma was ruptured. Time of injection was recorded from when the oocyte was in focus and the holding pipette entered the drop to when the oocyte was released from the holding pipette.

Invagination was scored based on previous literature Danfour, et. al. (2021), with 1 being the lowest and 3 being the highest, by estimating the level of oolemma invagination when injection pipette enters the oocyte at the 9 o'clock position for both treatments. Number of hand adjustments between controllers required for each single oocyte injection were quantified by video recordings. An adjustment was defined as an operator's hand leaving one controller and then using another separate controller.

For microarray tests, 10-20 matured oocytes were transferred into the microarray microwells containing 20 microlitre of HEPES media within the moat with a 170 micron ID flexi-pipette within a heated crib. All oocytes were maneuvered into the injection hemisphere within each microwell using the injection pipette and orientated to allow the 224 polar body to be at either 6 or 12 o'clock position. The time taken to orientate 10 oocytes was recorded and averaged on a per oocyte basis. Sperm were loaded into the injector as per conventional ICSI above, the injector entered the microwell through the injection slot, the injection was performed on the correctly orientated oocyte, and the injection pipette was drawn back through the slot, gently easing the pipette from the oocyte before gently pushing the oocyte back to the middle of the well with the pipette. The amount of time taken for injection during microarray procedure was recorded from when the oocyte and the injection pipette within the pipette slot was in focus until when the injection pipette was removed from the oocyte. Oocytes were excluded from culture if injection was unsuccessful for both convention ICSI or microarray ICSI.

Sham injections were conducted using microarray ICSI to assess parthenogenic activation and subsequent embryo development. For sham injections, oocytes were injected with 7% PVP and no sperm. Non-injected oocytes were also placed separately into culture as controls.

### Embryo Culture

Presumptive zygotes, sham injected, and controls were cultured in groups of 5 in 50 microlitre drops of modified NCSU-23 within a 60 mm petri dish (Falcon Product No: 351007) covered with ~8 mL of MEA-tested paraffin oil until Day 4 in a humidified incubator at 38.5 °C (6% CO2, 7% O2, balanced with N2). At Day 4 of culture, cleavage was scored, as well as post-injection survival rate and the degree of fragmentation in both un-cleaved and cleaved embryos. Fragmentation was classed to have occurred when greater than 30% of the perivitelline space was occupied by small, membrane-bound vesicular bodies. All surviving embryos were moved to equilibrated dishes with modified blastocyst NCSU-23 (ART Lab Solutions), supplemented with 10% FBS and overlayed with paraffin oil. Blastocysts were scored on Day 6 of culture.

### Differential Staining

Blastocysts produced from four replicates were collected for staining immediately after blastocyst scoring on Day 6. Blastocysts were washed in warmed (37 °C) HEPES-buffered NCSU23 for differential staining. The zona pellucidae were removed by incubation with 0.5% pronase dissolved in PBS for approximately 2 minutes, then washed in HEPES-MEM-PVA (Minimum Essential Medium and 1 mg/mL polyvinyl alcohol (PVA)) and then further incubation in 10 mM trinitrobenzenesulphonic acid (TNBS) in HEPES-MEM-PVA on ice for 15 minutes. Blastocysts were then removed and further washed in HEPES-MEM-PVA prior to being incubated with 0.2 mg/mL anti-dinitrophenol BSA in HEPES-MEM-PVA at 38 °C for 10 minutes. Blastocysts were then washed again in HEPES-MEM-PVA and incubated in HEPES MEM-PVA containing ~10% guinea pig complement serum, 0.01 mg/mL propidium iodide and 0.05 mM bisbenzimide (Hoechst 33258) for 10 minutes. After washing, blastocysts were transferred into HEPES-MEM-PVA as a post incubation step, then were dehydrated in 100% ethanol (Chem-Supply Pty Ltd, Gillman, SA, Australia) and mounted on a microscope slide in 2 microlitre of glycerol underneath a cover slip. Fluorescence images of the nuclei of blastocysts were captured with an epifluorescence inverted microscope (Eclipse TS100; Nikon Corporation; excitation filter 330-380 nm and barrier filter 420 nm). Numbers of blue inner cell mass (ICM) and pink to red trophectoderm (TD) cells were 265 counted post imaging by one researcher blinded to treatments.

### Human Microarray ICSI Validation

Vitrified human oocytes (n=18, from 2 patients) consented to research (The University of Adelaide Ethics Approval Number H-2023-082) were obtained from a local IVF clinic and warmed in the laboratory according to the SAGE vitrification warming kit instructions (Origio Australasia Pty Ltd). For C-ICSI, one 5 microlitre microdrop of 7% PVP (Origio Australasia Pty Ltd) was placed in the centre of a Vitrolife ICSI dish surrounded by eight drops 5 microlitre of GMOPS PLUS (Vitrolife Pty Ltd). The microarray dish comprised of one 5 microlitre strip of 7% PVP laid down the left side of the microarray, with 20 microlitre of GMOPS PLUS pipetted over and around the microarray moat. All ICSI dishes were overlayered with 5 mL of paraffin oil (Merck Group) and equilibrated to 37 °C for a minimum of 3 hours.

Oocytes were loaded into the conventional ICSI dish or microarray using a 170 micron Flexipet pipette tip as described for pig oocytes. Instead of sperm, 4 micron Tetraspeck microspheres (Thermofisher) that were centrifuged and washed in GMOPS PLUS medium were loaded into the 7% PVP droplets in each dish as a visual substitute for spermatozoon injection into human oocytes. A single bead was aspirated into the 5 micron ID injection pipette (Origio Australiasia Pty Ltd) from the PVP droplet. The injection process followed as per porcine conventional ICSI and microarray ICSI. The time for the bead injection and the number of hand adjustments were recorded as for porcine ICSI. Following successful injection, oocytes were observed for lysis after 90 minutes of culture. If un-lysed, they were reinjected to provide further timing and hand adjustment data. Immediately after the re-injection data was obtained, oocytes were discarded as biohazard material.

### Statistical Analysis

All statistical analysis was conducted using GraphPad Prism v9.5.1. All data were tested for normality to determine whether parametric or non-parametric analyses were applied. All binomial data was arcsin-transformed to make the data continuous for statistical comparison but is graphed as binomial data. Where appropriate, paired comparisons were used to account for replicate effects. The statistical test used, replicate numbers, sample sizes and p values are reported in each figure legend respectively. Data are presented as Mean+/−SEM and statistical significance was accepted at P<0.05.

### Results of Studies

### Effects on Preparation Time

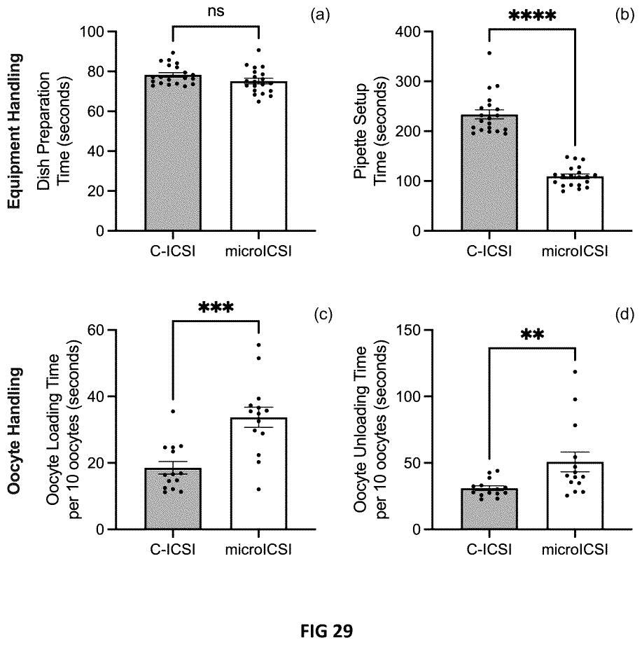

FIG. 29 (a) shows that dish preparation time for conventional ICSI and microarray ICSI were equivalent. However, at FIG. 29 (b) pipette setup time was reduced by half by removing the holding pipette. Loading the microwells added to oocyte handling time for both loading and unloading oocytes by 15-20 seconds (FIG. 29 (c) and (d)), but this was much less relative to the gain in time for pipette setup (>100 seconds).

### Effects on the ICSI Procedure

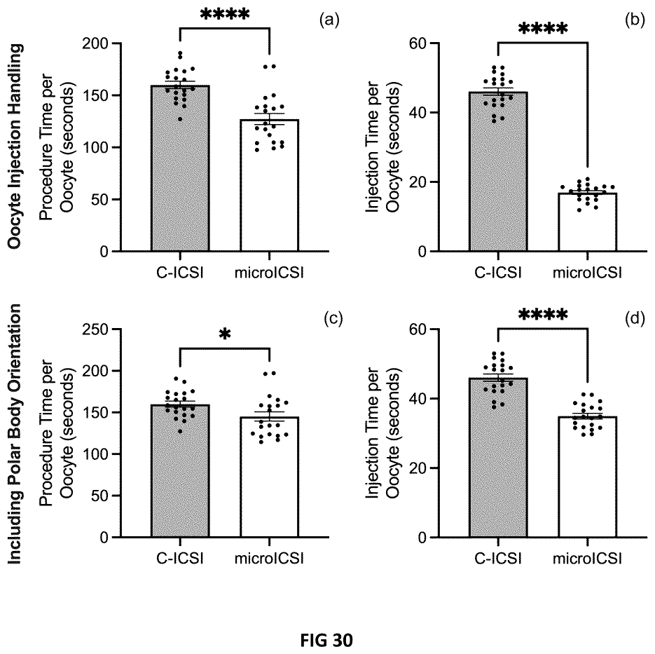

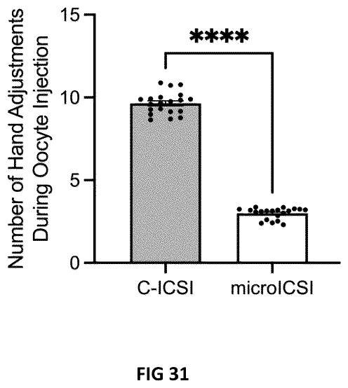

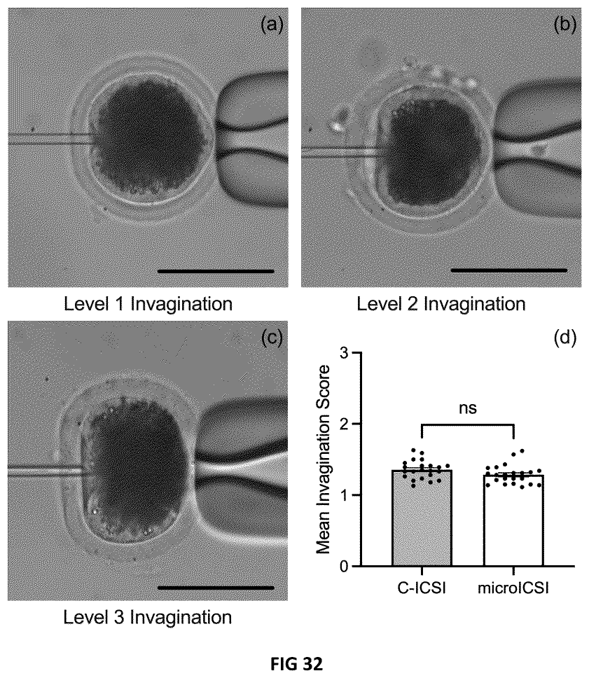

The average procedural time spent maneuvering and injecting each oocyte was reduced for microarray ICSI compared to conventional ICSI as shown in FIG. 30 (a). For the injection process, the time was more than halved for each oocyte (FIG. 30 (b)), and a reduction of time was still evident for microarray ICSI even when average polar body orientation time per oocyte was considered (FIGS. 30 (c) and (d)). Because the holding pipette was removed, the hand adjustments required to perform microarray ICSI were reduced by two thirds, as tracked by the number of hand adjustments between different controllers (FIG. 31). The level of invagination was equivalent between conventional ICSI and microarray ICSI (FIG. 32).

### Effect on Developmental Rate

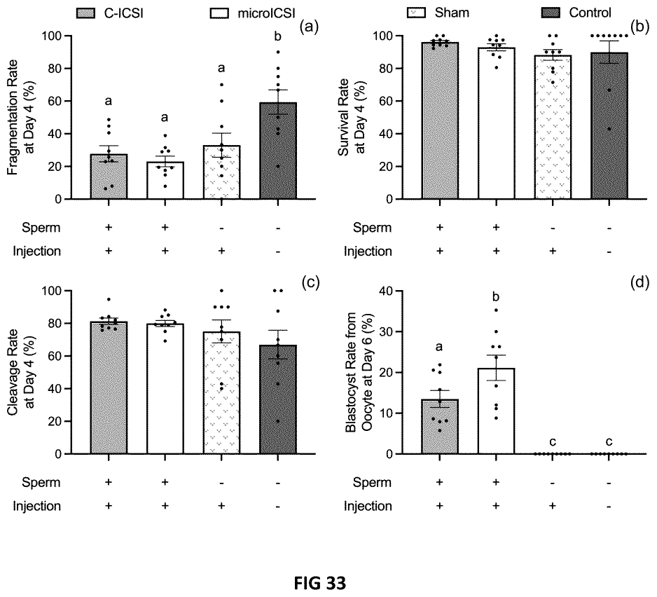

Rates of embryo development remained consistent between treatments, with fragmentation rate, survival rate and cleavage rates being equivalent across both ICSI techniques (FIG. 33 (a)-(c)). However, there was a significant increase in blastocyst rate for the microarray ICSI group compared to conventional ICSI (FIG. 33 (d)). Cell numbers of ICM and TD were equivalent between the blastocysts produced by conventional ICSI and microarray ICS, as shown in Table 1 below.

TABLE 1
Blastocyst cell numbers compared between
conventional ICSI and microarray ICSI

| | | Number of cells | | | ICM/TCN |
|---|---|---|---|---|---|
| Treatment | n | TD | ICM | TCN | (%) |
| C-ICSI | 16 | 27 +/− 2a | 7 +/− 1b | 34 +/− 3c | 19 +/− 2.5d |
| microICSI | 30 | 33 +/− 3a | 6 +/− 1a | 39 +/− 4 c | 16 +/− 1.8d |

### Validation of Translation to Human Oocytes

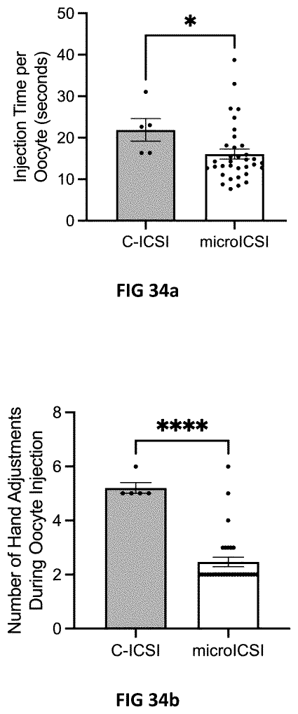

The microarray ICSI device was equally suited to perform microinjection on human oocytes after adjusting the hemisphere size. No immediate lysis was observed following the first round of bead injections and, from 18 oocytes, one oocyte was lysed after 90 minutes and excluded from re-injections. Consistent with porcine results, injection time and hand adjustments were reduced in microarray ICSI compared to conventional ICSI (FIGS. 34(a) and 34(b)).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be understood that the terms ‘fastener’ or ‘fastening’, ‘coupling’ or ‘sealing’ when used alone or together with other terms such as ‘means’ or others, may be used interchangeably where interpretation of the term would be deemed by persons skilled in the art to be functionally interchangeable with another. Further, the use of one of the aforementioned terms does not preclude an interpretation when another term is included.

The various apparatuses and components of the apparatuses, as described herein, may be provided in various sizes and/or dimensions, as desired. Suitable sizes and/or dimensions will vary depending on the specifications of connecting components or the field of use, which may be selected by persons skilled in the art.

It will be appreciated that features, elements and/or characteristics described with respect to one embodiment of the disclosure may be used with other embodiments of the invention, as desired.

Although the preferred embodiments of the present disclosure have been disclosed for illustrative purposes, those skilled in the art will appreciate that various modifications, additions and substitutions are possible, without departing from the scope and spirit of the disclosure and accompanying claims.

It will be understood that when an element or layer is referred to as being “on” or “within” another element or layer, the element or layer can be directly on or within another element or layer or intervening elements or layers. In contrast, when an element is referred to as being “directly on” or “directly within” another element or layer, there are no intervening elements or layers present.

As used herein, the term “and/or” includes any and all combinations of one or more of the associated listed items.

It will be understood that, although the terms first, second, third, etcetera, may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present disclosure.

Spatially relative terms, such as “lower”, “upper”, “higher”, “taller”, “top”, “bottom”, “left”, “right” and the like, may be used herein for ease of description to describe the relationship of one element or feature to another element(s) or feature(s) as illustrated in the figures. It will be understood that spatially relative terms are intended to encompass different orientations of structures in use or operation, in addition to the orientation depicted in the drawing figures. For example, if a device in the drawing figures is turned over, elements described as “lower” relative to other elements or features would then be oriented “upper” relative the other elements or features. Thus, the exemplary term “lower” can encompass both an orientation of above and below. The device may be otherwise oriented (rotated 90 degrees or at other orientations) and the spatially relative descriptors used herein should be interpreted accordingly.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the description are described herein with reference to diagrams and/or cross-section illustrations, for example, that are schematic illustrations of preferred embodiments (and intermediate structures) of the description. As such, variations from the shapes of the illustrations as a result, for example, of manufacturing techniques and/or tolerances, are to be expected. Thus, embodiments of the description should not be construed as limited to the particular shapes of components illustrated herein but are to include deviations in shapes that result, for example, from manufacturing.

Unless otherwise defined, all terms (including technical and scientific terms) used herein have the same meaning as commonly understood by one of ordinary skill in the art to which this description belongs. It will be further understood that terms, such as those defined in commonly used dictionaries, should be interpreted as having a meaning that is consistent with their meaning in the context of the relevant art and will not be interpreted in an idealised or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment of the description. The appearances of such phrases in various places in the specification are not necessarily all referring to the same embodiment. Further, when a particular feature, structure, or characteristic is described in connection with any embodiment, it is within the purview of one skilled in the art to effect and/or use such feature, structure, or characteristic in connection with other ones of the embodiments.

Embodiments are also intended to include or otherwise cover methods of using and methods of manufacturing any or all of the elements disclosed above.

While the invention has been described above in terms of specific embodiments, it is to be understood that the invention is not limited to these disclosed embodiments. Upon reading the teachings of this disclosure many modifications and other embodiments of the invention will come to the mind of those skilled in the art to which this invention pertains, and which are intended to be and are covered by both this disclosure and the appended claims.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It is indeed intended that the scope of the invention should be determined by proper interpretation and construction of the appended claims and their legal equivalents, as understood by those skilled in the art relying upon the disclosure in this specification and the attached drawings.

### CITATION LIST

Data on IVF clinics show wide variation in success rate, BMJ 2002; 325 doi: https://doi.org/10.1136/bmj.325.7362.460/e (Published 31 Aug. 2002).
Boulet, S. L., Meht, A., Kissin, D. M., Warner, L., Kawwass, J. F. and Jamieson, D. J. (2015) Trends in use of and reproductive outcomes associated with Intracytoplasmic Sperm Injection. Obstet Gynecol Surv. 70:325-6.
Palermo, G., Joris, H., Devroey, P. and Van Steirteghem, A. C. (1992) Pregnancies after intracytoplasmic injection of single spermatozoon into an oocyte. Lancet 340:17-18.
Joris, H., Nagy, Z., Van de Velde, H., De Vos, A. and Van Steirteghem, A. C. (1998) Intracytoplasmic sperm injection: laboratory set-up and injection procedure. Human Reproduction 13 Suppl. 1:76-86.
Latham, K. E. Stress signaling in mammalian oocytes and embryos: A basis for intervention and improvement of outcomes. Cell Tissue Res. 2016, 363, 159-167.
Gilchrist, R. B., Lane, M. and Thompson, J. G. (2008) Oocyte-secreted factors: regulators of cumulus cell function and oocyte quality Human Reproduction Update 14: 159-177.
Tiegs, A. W. and Scott, R. T. (2020) Evaluation of fertilization, usable blastocyst development and sustained implantation rates according to intracytoplasmic sperm injection operator experience. Reprod Biomed Online. 41:19-27.
Fernandez, J., Pedrosa, C., Vergara, F., Nieto, A. I., Quintas, A., et al. (2020) A new oocyte-holding pipette for intracytoplasmic sperm injection without cytoplasmic aspiration: An experimental study in mouse oocytes. Reprod Biol 20: 584-588.
Ma, S., Wang, P., Zhou, W., Chu, D. Zhao, S., Fu, l, and Li, F. (2020) A modified holding pipette for mouse oocyte fertilization. Theriogenology 141: 142-145.
Zander-Fox, D., Lam, K., Pacella-Ince, L., Tully, C., Hamilton, H., Hiraoka, K., et al. (2021) PIEZO-ICSI increases fertilization rates compared with standard ICSI: a prospective cohort study. Reprod Biomed Online. 43:404-12.
Danfour M. A., Elmahaishi M. S., (2010) Human oocyte oolemma characteristic is positively related to embryo developmental competence after ICSI procedure. Middle East Fertility Society Journal. 2010; 15:269-73.
Yagoub, S. H., Thompson, J. G., Orth, A., Dholakia, K., Gibson, B. C., and Dunning, K. R. (2022) Fabrication on the microscale: a two-photon polymerized device for oocyte microinjection. J Assist Reprod Genet. 2022; 39:1503-13.
Yagoub, S. H., Lim, M., Tan, T. C. Y., Chow, D. J. X, Dholakia, K, Gibson, B. C., Thompson, J. G., and Dunning, K. R. (2014) Vitrification within a nanoliter volume: oocyte and embryo cryopreservation within a 3D photopolymerized device. J Assist Reprod Genet. 2022; 39:1997-2014.
McLennan, H. J., Blanch, A. J., Wallace, S. J., Ritter, L. J., Heinrich, S. L., Gardner, D. K., Dunning, K. R., Gauvin, M. J., Love, A. K., and Thompson, J. G. Nano-liter perfusion microfluidic device made entirely by two-photon polymerization for dynamic cell culture with easy cell recovery. Sci Rep. 2023; 13:562.

1. A single cell handling device for handling a single cell during a microinjection procedure comprising;
a base dish having an upper surface and a lower surface,
a microwell array comprising one or more microwells, the one or more microwells comprising;
at least one microwell wall defining an upper microwell opening and a central well, the at least one microwell wall having formed therein two convex cell stabilizing protrusions each defining a cavity positioned opposite to the other on a common horizontal plane, the cell stabilizing protrusion comprising a first cell stabilizing protrusion defining a channel opening at a perimeter of an access channel extending outwardly through the microwell wall, and the second cell stabilizing protrusion defining a vent opening, the access channel having a channel base aligning with a centre point of the second cell stabilizing protrusion at a horizontal plane, and
a moat for maintaining fluid therein located at the base dish upper surface and extending around the perimeter of the microwell array.

2. A single cell handling device according to claim 1 wherein the moat is in fluid exchange with the one or more microwells via the access channel, the moat having an inner wall proximal to the microarray and an outer wall distal to the microarray, the outer wall extending vertically to a height taller than the channel base of the access channel and wherein the vent opening is entirely located higher than the channel base of the access channel.

3. A single cell handling device according to claim 1 wherein the first cell stabilizing protrusion and the second cell stabilizing protrusion is elliptically shaped in a horizontal plane.

4. A single cell handling device according to claim 3 wherein a diameter of the first cell stabilizing protrusion and a diameter of the second cell stabilizing protrusion are in the range of 10 microns to 500 microns.

5. A single cell handling device according to claim 3 wherein a diameter of the first cell stabilizing protrusion and a diameter of the second cell stabilizing protrusion are in the range of 100 microns to 200 microns.

6. A single cell handling device according to claim 1 wherein the at least one microwell wall is cylindrical in shape and the one or more microwells comprise a microwell base, wherein the at least one cylindrical microwell wall and the microwell base define the central well.

7. A single cell handling device according to claim 6 wherein the at least one cylindrical microwell wall has a diameter greater than the diameter of the cell stabilizing protrusions.

8. A single cell handling device according to claim 6 wherein the at least one cylindrical microwell wall has a diameter in the range of 0.1 microns to 1000 microns.

9. A single cell handling device according to claim 6 wherein the at least one cylindrical microwell wall has a diameter in the range of 200 microns to 300 microns.

10. A single cell handling device according to claim 1 wherein the access channel comprises two parallel channel walls, a channel base and an upper opening.

11. A single cell handling device according to claim 1 comprising a cover and one or more stacking connectors, located at an upper surface and at a lower surface so as to connect multiple base dishes when stacked.

12. A single cell handling device according to claim 6 comprising a sample support platform at the upper surface of the base plate having a sampling edge proximal to the access channel wherein the sampling edge is stepped or sloped at a gradient to align with the horizontal plane of the channel base.

13. A method of use of a single cell handling device according to claim 12 for performing an intracytoplasmic sperm injection procedure comprising the steps of;
a. obtaining a single cell handling device according to claim 1,
b. adding culture media to the moat of the single cell handling device,
c. obtaining an oocyte and a sperm sample,
d. adding the oocyte to the microwell central well,
e. placing the sperm sample on the sample support platform,
f. obtaining a microinjection apparatus,
g. aspirating a sperm from the sperm sample through a tip of the microinjection apparatus,
h. placing the tip through the access channel of the microwell,
i. shifting the oocyte to the second cell stabilizing protrusion with the tip,
j. microinjecting the oocyte with the sperm,
k. withdrawing the microinjection tip by cradling the oocyte in the first cell stabilizing protrusion while solely removing the microinjection tip.

14. A method of manufacture of a single cell handling device according to claim 1 comprising the fabrication of the base dish and fabricating the microarray and the moat thereon.

15. A method of manufacture of a single cell handling device according to claim 1 comprising the fabrication of the microarray and the moat within the base dish.

16. A method of manufacture according to claim 14 comprising a two part fabrication comprising a two photon polymerised printed part inserted into a polystyrene injection moulding.
